(19) United States
(12) Patent Application Publication
(10) Pub. No.: US 2025/0025330 A1
(43) Pub. Date: Jan. 23, 2025

(54) PROSTHETIC SPHINCTER APPARATUS

(71) Applicant: King's College London, london (GB)

(72) Inventors: Carlo SENECI-NIHR, London, Greater London (GB); Maleeha AL-HAMADANI, London, Greater London (GB)

(21) Appl. No.: 18/714,532

(22) PCT Filed: Nov. 23, 2022
(86) PCT No.: PCT/GB2022/052959

§ 371 Date: May 29, 2024

(30) Foreign Application Priority Data
Dec. 3, 2021 (GB) 2117473.5

(51) Int. Cl. A61F 5/44; A61F 5/445

(52) U.S. Cl. CPC A61F 5/4405; A61F 2005/4455

(57) ABSTRACT

A prosthetic sphincter apparatus comprising: a valve controllable between an open position for allowing a stool to pass through the valve and a closed position for preventing a stool from passing through the valve; and an implant configured to be surgically attached to a body part and to lock the valve inside the implant, wherein the valve is configured to be controllable between the open position and the closed position while remaining locked inside the implant.

### TECHNICAL FIELD

The present invention relates to a prosthetic sphincter apparatus and a method for attaching a prosthetic sphincter apparatus.

### BACKGROUND

There are number of different conditions that affect the bowel such as bowel cancer, ulcerative colitis, Crohn's disease and incontinence that may require a stoma as a necessary treatment. It is estimated that in the UK each year, around 14,000 patients undergo bowel resection, leading to large number of ostomic surgeries. A stoma is an opening created on the abdominal wall through the muscle and fat layers that can be connected to the digestive or urinary system and sutured onto the skin to divert the stool or urine, as the case may be, outside the body.

Since the stoma is an artificially created orifice without any natural control or sealing, a stoma bag is required for the collection of the stool that slowly but continuously leaves the body. The bag is attached to the skin via sticky tapes, which often cause skin blistering and discomfort to the patient and requires changing the bag two or three times daily. There are three types of stoma, including colostomy, ileostomy, and urostomy in which these three types can be either temporary or permanent for a time span that goes from a few months to a few years, usually to allow the bowel to recover after surgery. Often also patients who suffer from anal incontinence or who lost the use of the anus because of a resection due to bowel cancer recur to stomas.

Several scientific studies have shown that having a stoma can have a negative impact on people's daily lives including work, intimacy, exercise and quality of life in general. Because of the shortcomings of living with a stoma, some patients avoid undergoing stoma resection, even though their mortality risk is increased. Having a stoma does not only affect the patient physically, but also mentally. Young patients especially, tend not to socialise and in worst case become suicidal. Patients have to cope with physical and mental burdens and challenges on daily basis with managing a stoma. In general, the non-sealed stoma is secured to a bag to collect the diverted waste, and in some cases the bag moves out of its place, which results in leakage. Furthermore, leakage might result as the size of the fitting is bigger than the size of the stoma or the bag is loose and not stuck to the skin properly. In addition, if the bag contains air, it will start to detach from the skin, leading to leakage and odour issues, resulting in discomfort when changing the bag, especially in public. Patients also complain of blistering. The bag is fixed to the stoma using adhesive on the skin and as the bag is changed regularly during the day, the area where the adhesive is attached becomes irritated and blistering can occur. As a section of the bowel is sutured onto the skin, the delicate tissue of the bowel is exposed to the environment and contact with chemicals and dirt result in infection, which may require further surgery. The physical challenges also include the application and removal of the bag as it is difficult and painful to adhere or remove it from the skin. Moreover, cleaning the stoma inside and around the stoma becomes challenging for some patients as they don't feel comfortable touching the stoma or surrounding area, especially during the early stages after a fitting. Having a stoma and carrying a stoma bag become an obstacle during physical activities, for example patients have complained that the adhesive becomes loose during physical activities. The adhesive detaches from the edges during activities, such as sport and swimming. Some patients participate in social events that require them to travel for long distances and thus require an extended period wearing the bag without leaking and production of unpleasant odour. Recent research included the improvement of stoma bags to be sealed firmly, by attaching a protective seal to prevent leakage and if the patients have skin creases or folds as a result of excess fat. A powder is used to remove sweat and moisture to reduce irritation of the skin surrounding the stoma site. However, while still having to use a bag, leakage, bad odour, blistering and irritation can still occur. Patients also complain about other issues whilst wearing a bag, such as the choice of clothing being limited to bigger sizes to hide the bag and not being able to carry out daily activities without having to worry about when and where to empty and change it.

Moreover, the stoma appearance is often considered disturbing by the patient who are exposed to an additional psychological burden due to its aesthetics. More recent studies have focused on removing the need for a bag by designing a stoma valve that is able to be sealed, thereby, eliminating leakage and bad odour.

For example, US 2013/0060212 A1 discloses a stomal insert for conducting waste content from an intestinal portion in an abdominal cavity through a stoma in an abdominal wall comprising; a cover for covering the stoma; a fixation element for anchoring the stomal insert to a visceral side of the abdominal wall; and a pliable, axially elastic tube interconnecting said cover and said fixation element and sized to apply a tensile force to said cover and to said fixation element.

As another example, US 2012/0289916 A1 discloses a method of providing a stoma with a removable connection for a receptacle includes securing an implant onto the stoma of a patient, wherein the implant has an axial interior section for fixation inside the body and an axial exterior section extending outwards from the body, a free end of the exterior section accommodates mounting of a detachable device, and a distal portion of the interior section includes an anchoring section, extending radially from the distal portion of the interior section. The anchoring section has an inner anchoring ring, an outer anchoring ring and at least one connection member configured and dimensioned for resiliently connecting the inner and outer anchoring rings in a manner to provide axial resilience and anchorage to the anchoring section such that the anchoring section is able to respond to movements and absorb the shear of the implant in relation to adjacent organs.

Meanwhile, US 2021/0121317 A1 discloses a lid for an ostomy implant that comprises: attachment means for attachment to the implant; and an opening part. The opening part is arranged such that it can be opened and closed while the lid is attached to the implant.

The current state of the art fails to produce a solution that overcomes the aforementioned complications. These examples all rely on the stoma being formed according to known stoma forming surgery, having the bowel on the outside.

It is desirable to improve the physical and psychological condition of the patient by providing them with a prosthetic valve that improves their quality of life. It is desirable to provide a device that reduces leakage and odours, may provide a warning and that allows a patient to release waste material more easily in a controlled manner, whilst eliminating the risk of infection and skin blistering.

### SUMMARY OF INVENTION

According to an aspect of the present invention there is provided a prosthetic sphincter apparatus comprising: a valve controllable between an open position for allowing waste to pass through the valve and a closed position for preventing waste from passing through the valve; and an implant configured to be surgically attached to a body part and to lock the valve inside the implant, wherein the valve is configured to be controllable between the open position and the closed position while remaining locked inside the implant.

According to another aspect of the present invention there is provided a method for attaching a prosthetic sphincter apparatus, the method comprising: surgically attaching an implant to a body part, the implant configured to lock a valve inside the implant, the valve being controllable between an open position for allowing a stool to pass through the valve and a closed position for preventing a stool from passing through the valve, wherein the valve is configured to be controllable between the open position and the closed position while remaining locked inside the implant.

### DRAWINGS

The present invention will be described by way of example only with reference to the accompanying drawings.

FIG. 1 is a schematic diagram of a prosthetic sphincter apparatus according to an embodiment of the invention.

FIG. 2 is a cross-sectional view of the prosthetic sphincter apparatus shown in FIG. 1.

FIG. 3 is a perspective view of an inner mould for a valve of a prosthetic sphincter apparatus.

FIG. 4 is a view of an outer mould for a valve of a prosthetic sphincter apparatus.

FIG. 5 is an axial view of a prosthetic sphincter apparatus.

FIG. 6 is an expanded view of one end of a prosthetic sphincter apparatus.

FIG. 7 is a cross-sectional view of an implant of a prosthetic sphincter apparatus.

FIG. 8 is another view of the implant shown in FIG. 7.

FIG. 9 is a perspective view of a prosthetic sphincter apparatus.

FIG. 10 is a perspective view of a valve of a prosthetic sphincter apparatus.

FIG. 11 is an axial view of an alternative valve of a prosthetic sphincter apparatus.

FIG. 12 is a perspective view of the valve shown in FIG. 11.

FIG. 13 is a perspective view of a further alternative valve of a prosthetic sphincter apparatus.

FIG. 14 is a perspective view of the valve shown in FIG. 13 in the open position.

FIG. 15 is a perspective view of a further alternative valve of a prosthetic sphincter apparatus.

FIG. 16 is a cross-sectional view of a prosthetic sphincter apparatus.

FIG. 17 is a cross-sectional view of an implant of a prosthetic sphincter apparatus.

### DETAILED DESCRIPTION

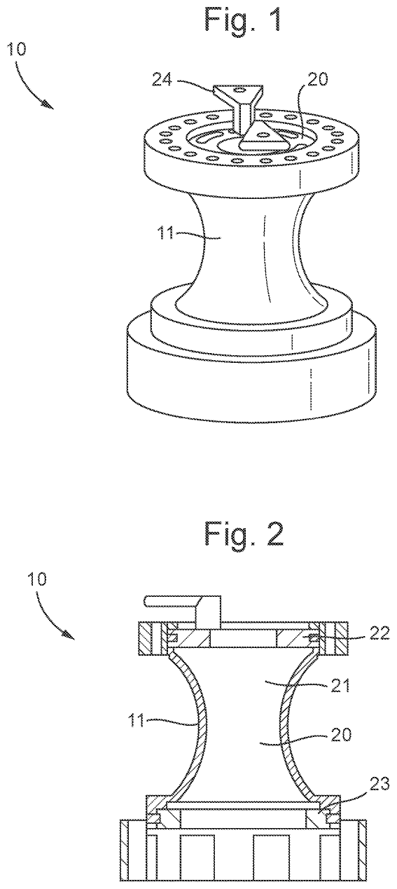

FIG. 1 shows a prosthetic sphincter apparatus 10 according to an embodiment of the invention. As shown in FIG. 1, the apparatus 10 comprises a valve 20. The valve 20 is controllable between an open position and a closed position. The open position is for allowing waste to pass through the valve 20. The closed position is for preventing waste from passing through the valve 20. The waste may be material such as a stool.

FIG. 2 shows a cross-sectional view of the apparatus 10 shown in FIG. 1. In the view shown in FIG. 2, the valve 20 is in the open position. When the valve 20 is in the open position, a channel is open from one end of the valve 20 to the other end of the valve 20. The waste can pass through the open channel.

As shown in FIG. 1, the apparatus 10 comprises an implant 11. The implant 11 is configured to be surgically attached to a body part 30 of a patient. For example, the implant 11 may be configured to be attached to the abdomen of a patient. The implant is configured to lock the valve 20 inside the implant 11. For example, the valve 20 and the implant 11 may comprise one or more complementary elements that engage with each other so as to fix the location of the valve 20 relative to the implant 11. When the valve 20 is fixed relative to the implant 11, the valve 20 is inside the implant 11.

As shown in FIG. 1 and FIG. 2, the apparatus 10 may extend along an axial direction. The implant 11 is configured to round the valve 20. The implant 11 is radially outward of the valve 20. When viewing the apparatus 10 along the axial direction, the implant 11 surrounds the valve 20.

The implant 11 may comprise one or more holes suitable for allowing the implant 11 to be attached or fixed to the body part 30. For example, sutures may extend through the holes of the implant 11 so as to fix the implant 11 in position relative to the body part 30. Alternatively, the implant 11 may be adhered to the body part 30, stapled to the body part 30 and/or mechanically locked to the body part 30. The implant 11 is configured to remain attached to the body part 30 when in use of the apparatus 10. The external side of the implant 11 may be stitched onto the skin 32. The implant 11 may comprise slots in the middle and towards one or both ends. The bowel 31 may be sutured onto the implant 11 through one or more of the slots. The muscle 34 may be sutured onto one or more slots nearer the end of the implant 11 that faces into the body.

The valve 20 may be removed from the implant 11. The valve 20 may be unlocked from the implant 11 and removed from the patient. This allows the valve 20 to be maintained and/or cleaned, for example. The valve 20 may be configured to be inserted and removed for maintenance or substitution without requiring the need for a surgery. The valve 20 may comprise a mechanical mechanism for locking the valve 20 into the implant 11 securely, unless it is intended to be removed. For example, if the patient is required to undergo an MRI examination, the valve 20, which may contain sensors and/or actuators as described below, can be removed and another valve 20 or a cap can be inserted instead during the scan. The apparatus 10 may be an MRI compatible device.

The valve 20 is configured to be controllable between the open position and the closed position while remaining locked inside the implant 11. It is not necessary for the valve 20 to be removed from the implant 11 or from the body part in order for the waste to be removed from the patient. While the valve 20 remains locked in the implant 11, the valve 20 may be controlled from the closed position to the open position, thereby allowing the waste to pass through the valve 20 and exit the body of the patient. The invention is expected to make it easier for the patient to controllably remove waste from their body. After waste removal, the valve 20 may be controlled to return to the closed position.

For the purposes of waste removal, optionally a bag holder is provided to manually fix onto the valve 20. The bag holder may be provided with a sustainable, flushable bag to remove waste.

As shown in FIG. 1, optionally the apparatus 10 comprises a handle 24. The handle 24 may be part of the valve 20. The handle 24 is configured to allow the patient to manually control the valve 20 between the open position and the closed position. For example, the patient may rotate the handle 24 as to switch between the open position and the closed position of the valve 20. It is not essential for the handle 24 to be provided. Additionally or alternatively, the valve 20 may be controlled remotely. The valve 20 may comprise a mechanism to bring the valve 20 between the open position and the closed position, the mechanism capable of being controlled remotely.

Optionally, the valve 20 and the implant 11 are configured such that the valve 20 can be repeatedly removed from and reinserted into the implant 11. The locking mechanism for locking the valve 20 into the implant 11 is separate from the mechanism for switching the valve 20 between the open position and the closed position. The valve 20 may be removed so as to allow maintenance to be performed on the valve 20, or to allow the valve 20 to be cleaned, for example.

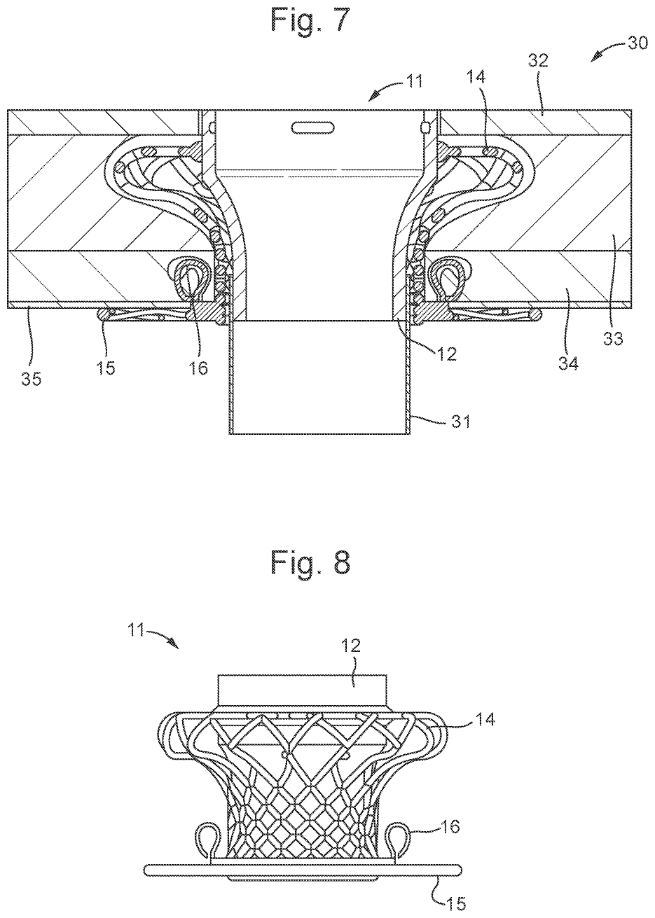

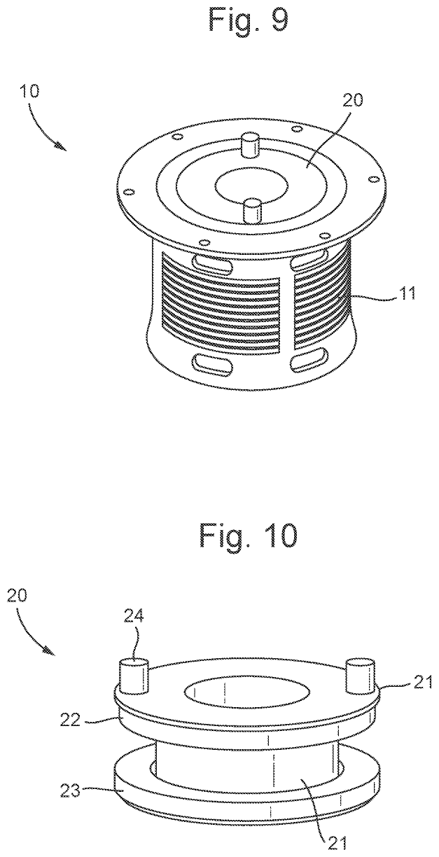

FIG. 7 is a cross-sectional view of an implant 11 according to an embodiment of the invention. FIG. 7 shows the implant attached to the body part 30 which may be part of the abdomen of a patient. As shown in FIG. 7, optionally a plurality of holes are defined in the implant 11. The holes are for allowing the implant 11 to be sutured to the body part 30. In the example shown in FIG. 7, holes are defined in one or more meshes. Any meshes of the implant 11 may be bioabsorbable. Additionally, or alternatively, as shown in FIG. 9, for example holes may be defined in the main body 12 of the implant 11 (FIG. 9 showing sets of holes at either end of the implant 11). One or more sutures may pass through the holes and through the body part 30 so as to fix the implant 11 relative to the body part 30. The valve 20 is locked relative to the implant 11 during use of the apparatus 10. It is not necessary to provide any further device such as a strap to hold the valve 20 in place relative to the body part 30. The apparatus 10 may be more comfortable for patients to wear.

As shown in FIG. 7, optionally the plant 11 comprises a flange 14. The flange 14 protrudes radially outwards. The flange 14 is for anchoring the implant 11 relative to the body part 30.

As shown in FIG. 7, optionally the flange 14 comprises a mesh. The mesh of the flange 14 protrudes into the fat 33 of the body part 30 so as to anchor the implant 11 in place. Optionally, the mesh is sutured to the fat 33 using dissolvable sutures. Over time the sutures may dissolve, by which point they may no longer be needed. Further, it is not essential for the flange 14 to comprise a mesh. For example, alternatively the flange 14 may form as a circular flat disc as shown in FIG. 9. The mesh structure may not be required. This can help to reduce the possibility of any mesh damaging the body part 30.

The layer of fat 33 may protrude through the mesh in order for the implant 11 to be fixed in place. As shown in FIG. 7, optionally the implant 11 is configured such that there is a gap between the main body 12 and the mesh. The main body 12 may be flexible or rigid. The bowel 31 can be pulled through and the mucosa can be stitched onto the skin 32. Optionally the mesh is made of a dissolvable material. During the healing of the bowel 31 and the skin 32, part of the mesh may dissolve, leaving only the main body 12 fixed onto the bowel 31.

Optionally, the flange 14 is located so as to be positioned between the skin 32 and the fat 33 of the body part. Alternatively, the flange 14 may be arranged to be positioned externally of the skin 32. When the flange 14 is formed as a vertical disc as shown in FIG. 9, optionally the flange 14 comprises ridges. The ridges are for creating grip with the fat 33.

The implant 11 is made of a biocompatible material. Optionally, the implant 11 is ridged. For example, the implant 11 may comprise a metal such as titanium. Alternatively, the implant 11 may be configured to be flexible. The implant 11 may be made of a flexible material such as nylon, silicon or a compliant titanium structure. The implant 11 may comprise a biocompatible and long-term lasting rigid material such as titanium, nylon or PEEK or a soft and flexible material such as silicone or PDMS.

As shown in FIG. 7, the implant 11 may comprise a main body 12. The main body 12 may define an axial direction and cavity for housing the valve 20. Optionally the implant 11 comprises external ridges. The external ridges may be for gripping the implant 11 to fat 33, for example. The external ridges may be for gripping the implant 11 to other tissues such as muscle. The external ridges may reduce the possibility of the implant 11 undesirably moving (e.g. rotating) relative to the body part 30 during use.

As shown in FIG. 7, optionally the implant 11 comprises hooks 16. The hooks 16 are for allowing the implant 11 to be sutured to the body part 30. For example, the hooks 16 may be for allowing the implant 11 to be sutured to ligaments (e.g. ligaments between muscle and fat) and/or muscle 34 of the body part 30. The hooks 16 may define the holes mentioned above as being defined in the implant 11 for allowing the implant 11 to be sutured to the body part 30. As shown in FIG. 7, optionally, the hooks 16 are positioned close to the internal end of the implant 11. The internal end means the end (in the axial direction) of the implant 11 that is at the internal side of the body part 30. The other end of the implant 11 may be called the external end of the implant 11. The external end of the implant 11 faces away from the patient. By providing the hooks 16 near the internal end of the implant 11, the hooks 16 may be used for fixing the implant 11 to the ligaments and/or muscle 34 and/or peritoneum 35 of the abdomen wall.

Optionally, the implant 11 is configured such that the bowel 31 can be stitched to the implant 11. For example, the bowel 31 may be stitched to the implant 11 via holes defined in the main body 12 of the implant 11. As shown in FIG. 7, optionally the bowel 31 extends only part way through the abdomen wall. It is not necessary for any of the bowel 31 to reach the exterior of the body part 30. This can reduce the possibility of the bowel 31 being damaged, for example by irritation or infection. As shown in FIG. 7, optionally the end of the bowel 31 is in the muscle 34 of the body part 30. Alternatively, the end of the bowel 31 may reach into the fat 33. Optionally, the bowel 31 may reach to the skin 32 and may be stitched to the skin 32, for example. The present invention may provide an implantable valve that can replace stomas and/or will allow for anus reconstructive surgery. The same valve would be suitable to be implanted in lieu of a stoma or by replacing the anus of a patient.

Normally a stoma is a non-controllable orifice and the stool is stored outside of the patient's body. The apparatus 10 may be combined with use of the J-pouch colostomy surgical approach to create an internal pouch where the stool can accumulate before being released from the body instead of using an external bag. An advantage is to ease exit of the stool and reduction of hydration loss and minerals. The apparatus 10 may be connected to the J-pouch to direct the stool outside of the body in a controlled manner.

As shown in FIG. 7, optionally, the implant 11 comprises a skirt 15. The skirt may be formed as a flange, for example protruding radially outwards from the main body 12 of the implant 11. The skirt 15 is for anchoring the implant 11 to the body part 30. For example, the skirt 15 may be for anchoring the implant 11 to the peritoneum 35. The skirt 15 may be stitched and/or stapled to the peritoneum 35. The skirt 15 may be positioned near the internal end of the implant 11. The skirt 15 may reduce the possibility of the implant 11 undesirably moving (e.g. in the axial direction) during use of the apparatus 10 and prevent hernia formation.

As shown in FIG. 7, optionally the skirt 15 comprises a mesh. Alternatively, the skirt 15 may be formed as a flat disc. As a further alternative, the skirt may not be provided, for example as shown in FIG. 9. As shown in FIG. 9, optionally the main body 12 of the implant 11 flairs outwards towards the internal end of the implant 11. The flaring may help to anchor the implant 11 in place, reducing the possibility of the implant 11 moving towards the exterior of the body part 30 during use.

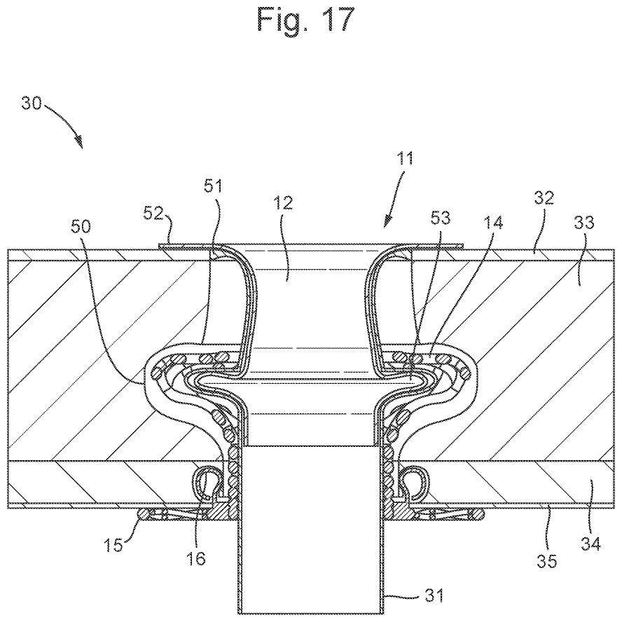

FIG. 17 is a cross-sectional view of an implant 11 according to an embodiment of the invention. The implant 11 shown in FIG. 17 is different from the implant 11 shown in FIG. 7. However, the implant 11 shown in FIG. 17 shares features in common with the implant 11 shown in FIG. 7. The features in common are not described below so as to avoid repetition. Features of the implant 11 shown in FIG. 17 that are not shown in FIG. 7 are described below.

As shown in FIG. 17, optionally the implant is provided at a hole in the skin 32. Optionally, the bowel 31 is attached to the inner surface 51 of the skin that defines the hole. For example, the bowel 31 may be stitched to the skin 32. The bowel 31 and the skin 32 may subsequently grow into each other over time. The bowel 31 may not protrude outwardly beyond the skin 32.

As shown in FIG. 17, optionally the implant 11 comes in two pieces. In particular the main body 12 of the implant may be formed as a separate piece from an attachment assembly 50. The implant 11 comprises the main body 12 and the attachment assembly 50. The attachment assembly 50 is for fixing the implant 11 to the body part 30. For example, the attachment assembly 50 may comprise the flange 14 and the skirt 15. The main body 12 comprises the tube into which the valve 20 is inserted.

The flange 14 and/or the skirt 15 may comprise meshes. The meshes may be a separate part from the implanted tube. Optionally, the attachment assembly 50 is inserted by a medical procedure. Subsequently, the main body 12 may be inserted. For example the main body 12 may be added six weeks after the medical procedure for fixing the attachment assembly 50 to the body part 30. The main body 12 may be added without requiring any surgery. An embodiment of the invention is a method that is performed after the attachment assembly 50 has been attached to the body part 30. The method comprises attaching the main body 12 of the implant 11 to the attachment assembly 50. The method does not involve any surgical step.

As shown in FIG. 17, optionally the main body 12 comprises an external flange 52. The external flange is configured to be located at the external side of the skin 32. The external flange 52 may not significantly protrude outwards from the skin 32. It is desirable for the external flange 52 to be as flush as possible against the skin 32. The external flange 52 is configured to anchor the main body 12 to the skin 32. The external flange 52 reduces the possibility of the main body 12 moving further into the body of the patient.

Optionally, the external flange 52 is fixed to the skin 32. For example, an adhesive may be provided for adhering the external flange 52 to the skin 32.

As shown in FIG. 17, optionally the main body 12 comprises a radial extension 53. The radial extension 53 protrudes radially outwards. The radial extension 53 may form a flange or a flange-like member. The radial extension 53 may be configured such that at least part of the radial extension 53 extends into a pocket formed by the flange 14. The radial extension 53 may be configured to reduce the possibility of axial movement of the main body 12 relative to the attachment assembly 50. The radial extension 53 and the external flange 52 may combine to anchor the main body 12 in place.

As shown in FIG. 17, optionally the bowel 31 is between the main body 12 and the attachment assembly 50 of the implant. The bowel 31 may separate the main body 12 from the attachment assembly 50. The main body 12 may not contact the attachment assembly 50. Nevertheless the main body 12 engages indirectly with the attachment assembly 50, for example by the radial extension 53 in the pocket of the flange 14 of the attachment assembly 50.

FIG. 8 is a view of the exterior of the implant 11. As shown in FIG. 8, optionally the mesh of the flange 14 extends from the flange 14 in the axial direction towards the internal end of the implant 11. Optionally, the mesh of the flange 14 is integral with the hooks 16 and/or the skirt 15. Alternatively, the flange 14 may be formed as a mesh which is separate from the hooks 16 and/or the skirt 15 (albeit in position relative to the hooks 16 and the skirt 15 through the main body 12 of the implant 11).

As shown in FIG. 2, optionally the implant 11 defines a fixed tube having an hourglass shape. The fixed tube may be formed by the main body 12 of the implant 11. The hourglass shape means that the cross-sectional area of the fixed tube is greater at the internal and external ends of the implant 11 compared to at an intermediate point between the internal and external ends. As shown in FIG. 2, optionally the cross-sectional area of the fixed tube of the main body 12 changes gradually along the axial direction of the implant 11. The hourglass shape may help to hold the implant 11 in position relative to the body part 30.

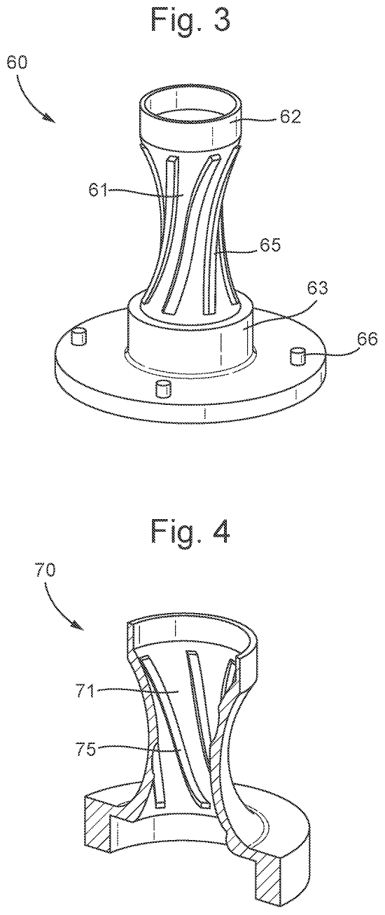

FIG. 3 is a perspective view of an inner mould 60 of a valve 20. Half of outer mould 70 cut away from the other half is shown in FIG. 4. The inner mould 60 and the outer mould 70 combine so that the valve 20 can be formed. As shown in FIG. 2, optionally the valve 20 comprises a flexible tube 21. The flexible tube 21 defines a channel through which the waste may pass. As shown in FIG. 3, optionally the inner mould 60 comprises an inner wall 61 configured to define the inner surface of the flexible tube 21. As shown in FIG. 4, optionally the outer mould 70 comprises an outer wall 71 configured to define the outer surface of the flexible tube 21.

As shown in FIG. 3, optionally the inner mould 60 comprises locking elements 66. The locking elements 66 are configured to lock the inner mould 60 relative to the outer mould 70 shown in FIG. 4. For example, as shown in FIG. 3, optionally the locking elements 66 comprise pins. The pins are configured to slot into complementarily shaped slots defined in the outer mould 70. As shown in FIG. 3, optionally the inner mould 60 comprises a flange attached to the wall 63 defining the inner surface of the internal ring 23. The flange comprises the locking elements 66. The inner mould 60 further comprises a wall 62 defining the inner surface of the external ring 22. Optionally the flexible tube 21 of the valve 20 comprises ridges. The ridges may extend in the axial direction. The ridges may also extend at least partly in the circumferential direction. The ridges may be formed between inner ridge surfaces 65 of the inner mould 60 and outer ridge surfaces 75 of the outer mould 70.

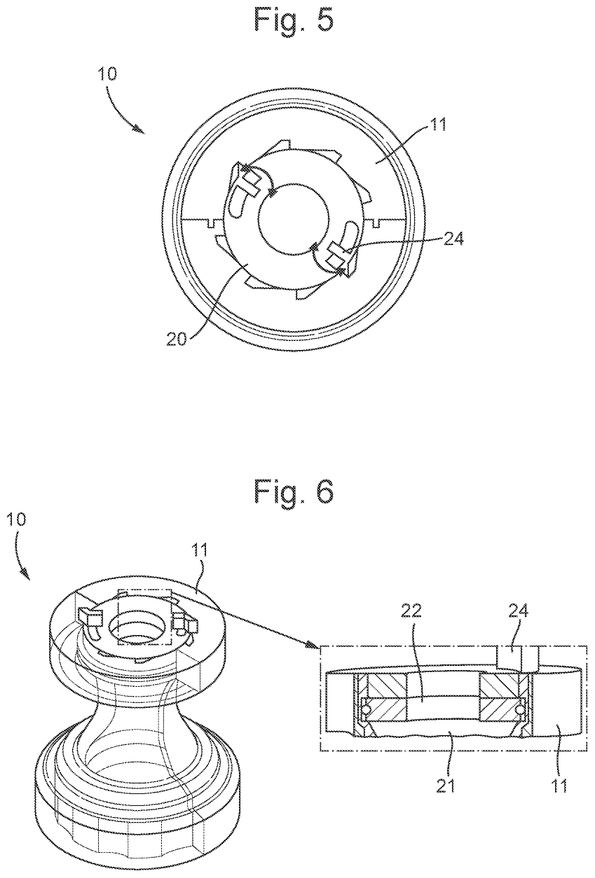

FIG. 5 is an axial view of the apparatus 10. FIG. 5 shows the external ends of the implant 11 and the valve 20. As shown in FIG. 5, the valve 20 is surrounded by the implant 11. The external end of the valve 20 may comprise a handle 24. The handle is for rotating the external end of the valve 20. The handle 24 is for controlling the valve 20 between the open position and the closed position.

FIG. 6 is an expanded view of the external end of the valve 20 locked in the implant 11. As shown in FIG. 6, the handle 24 is relative to the external ring 22. When the handle 24 is operated, the external ring 22 rotates relative to the implant 11. As shown in FIG. 6, optionally the external ring 22 comprises elements such as an O-ring configured to rotate relative to a groove formed in the internal wall of the external end of the implant 11. The external ring 22 is configured to allow rotation relative to the implant 11 without allowing axial movement of the external ring 22 relative to the implant 11.

The valve 20 is a functioning element of the apparatus 10. The valve 20 carries a valve mechanism. The valve mechanism may come in different configurations as described below.

Optionally the valve mechanism is a twisting mechanism, in which, for example, a cylindrical silicone sheet is attached to a ring from both sides and when twisted, the valve 20 is closed. Optionally the flexible tube 21 is arranged such that twisting of one end 22 of the flexible tube 21 relative to the other end 23 of the flexible tube 21 causes the valve 20 to be controlled between the open position in which the channel is open and the closed position in which the channel is at least partly closed.

The valve 20 is configured such that the ends of the flexible tube 21 can be rotated relative to each other. One end may be the external end of the flexible tube 21. The other end may be the internal end of the flexible tube 21. The internal end is the end that the internal side of the body part 30 when the apparatus 10 is in use. The external end is the end that is at the external side of the body part 30 when the apparatus 10 is in use.

As shown in FIG. 2, for example, optionally the valve 20 comprises an external ring 22 attached to one end of the flexible tube 21. The external ring 22 is configured such that rotation of the external ring 22 causes the end of the flexible tube 21 to which the external ring 22 is attached to be rotated relative to the other end. While the valve 20 remains locked in the implant 11, the external ring 22 can be rotated relative to the implant 11.

Optionally, the valve 20 comprises an internal ring 23 attached to one end of the flexible tube 21. The internal ring 23 is configured to be fixed relative to the implant 11 when the valve 20 is locked inside the implant 11. The internal ring 23 cannot be rotated relative to the implant 11 during use of the apparatus 10. When the external ring 22 is rotated, the internal ring 23 remains in a fixed position relative to the implant 11 and the body part 30. Alternatively, the internal ring 23 may not be provided. Instead, the end of the flexible tube 21 may be fixed directly onto the implant 11 or it may be directly part of the main body 12.

When the ends of the flexible tube 21 are rotated relative to each other, the flexible tube 21 of the valve 20 twists. The twisting reduces the cross-sectional area of the channel that the flexible tube 21 defines. The cross-sectional area is reduced at an intermediate point between the ends of the flexible tube 21. Reduction in cross-sectional area helps to block waste material from passing through the channel defined by the flexible tube 21.

The twisting mechanism may be formed from two rings with a silicone cylindrical/hourglass shaped tube attached in the middle, in which the silicone is twisted to seal the implant. This is a particularly simple design with a full passage when opening the valve 20. This enables easy use of the apparatus 10 and easy cleaning of the valve 20 by patients. Once the patient is in the process of excreting the waste, a bag shutter is attached and is rotated to untwist the bag, thus, releasing the waste.

Optionally, the apparatus 10 is arranged such that the ends of the flexible tube 21 can be rotated at least one full rotation relative to each other. By one full rotation, the cross-sectional area of the channel may be reduced substantially. Of course, it is possible that some waste material such as liquid or a gas may pass through the closed valve 20. Optionally, the apparatus 10 is arranged such that the ends of the flexible tube 21 can be twisted at least one and a half full rotations, and optionally at least two full rotations relative to each other. By increasing the amount of rotation between the ends of the flexible tube 21, the possibility of waste material undesirably leaking through the closed valve 20 is reduced. In particular, it has been found that two full rotations reduces any undesirable leaking by more than 99% (relative to one single full rotation).

As shown in FIGS. 2 to 4, for example, optionally the flexible tube 21 has an hourglass shape. The cross-sectional area of the channel defined by the flexible tube 21 greater at either end of the flexible tube 21 than at an intermediate position between the ends. The flexible tube 21 of the valve 20 may be shaped as to be complementary to the inner surface of the main body 12 of the implant 11.

FIG. 10 is another schematic perspective view of the valve 20. As shown in FIG. 10, optionally the flexible tube 21 extends through the external ring 22 and engages with the handle 24. One end of the flexible tube 21 is fixed relative to the handle 24. Rotation of the handle 24 causes the flexible tube 21 to twist, closing its cross-sectional area.

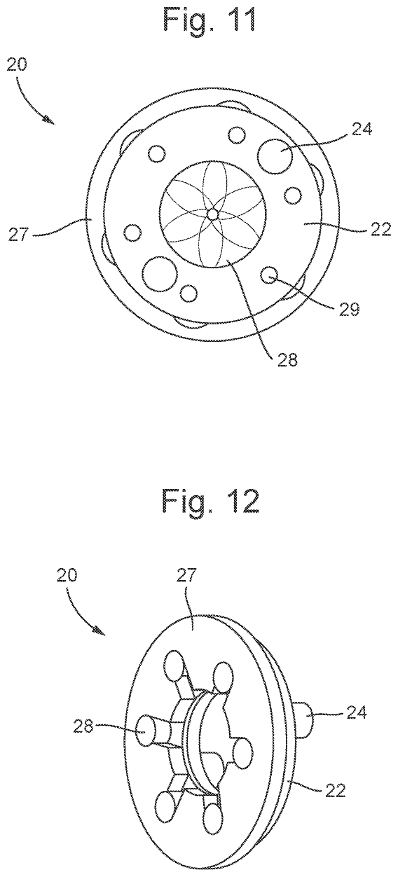

FIG. 11 is an axial view of a valve 20 with an alternative valve mechanism. FIG. 12 is a perspective view of the valve 20 shown in FIG. 11. FIG. 11 shows the valve 20 in the closed position. FIG. 12 shows the valve in the open position. The valve 20 shown in FIGS. 11 and 12 comprises an iris mechanism. The iris mechanism may comprise a plurality (e.g. six) blades 28, a base plate, and a blade actuating ring as described below.

Optionally, the valve 20 comprises moveable components configured to be moveable between a first position corresponding to the open position and a second position corresponding to the closed position. In the second position corresponding to the closed position, the moveable components that at least partly block the channel through the valve 20. In the example shown in FIGS. 11 and 12, the moveable components comprise blades 28 of an iris mechanism. FIG. 12 shows the blades 28 in the first position. FIG. 11 shows the blades 28 in the second position. As can be seen in FIG. 11, the blades 28 at least partly block the channel through the valve 20. Optionally, the blades 28 completely block the channel through the valve 20 in the second position.

As shown in FIGS. 11 and 12, optionally the blades 28 are provided between the external ring 22 and a housing ring 27 (which may be referred to as a base plate and a blade actuating ring). The blades 28 may be fixed to the external ring 22 and/or the housing ring 27 via a pivot 29. The pivot 29 may be formed as a pin that can rotate within a complementarily shaped hole in the external ring 22. The external ring 22 may be rotated, for example by operation of the handle 24. When the external ring 22 is rotated, the blades 28 are controlled between the first position and the second position. The blades 28 may be configured to move in a synchronised way.

The iris mechanism may be similar to the lens shutter of a camera, which when closed, no light can pass through. Optionally, a silicone tube with two disks on either side is inserted between the blades 28 to reduce leaking in case the blades 28 are not able to fully seal the valve 20.

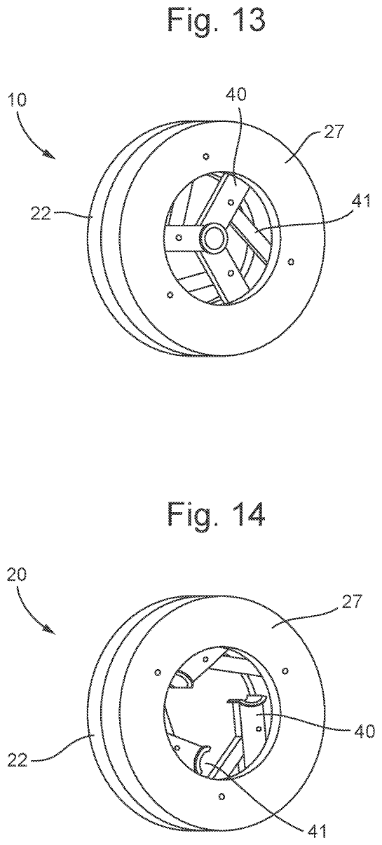

FIG. 13 is a perspective view of an alternative valve 20 in the closed position. FIG. 14 shows the valve of FIG. 13 in the open position. In the examples shown in FIGS. 13 and 14, the moveable components, which are moveable between the first position and the second position, comprise hinged rods 40 of a hinge mechanism. The hinge mechanism may be called a three hinges mechanism.

The hinged rods 40 may be located between the external ring 22 and the housing ring 27 of the valve 20. The hinged rods 40 may comprise struts 41 for connecting the hinged rods 40 to the external ring 22 and the housing ring 27. The struts 41 may be connected to the external ring 22 and the housing ring 27 via respective pivots. The struts 41 are configured to pivot so as to control movement of the hinged rods 40 between the first position and the second position. In the second position, the hinged rods 40 partly block the channel through the valve 20.

A sheet of flexible material such as silicone may be attached to the housing ring 27 and into the three hinged rods 40 in the middle. When the rings are rotated, the hinges open the valve 20, thus allowing waste to be excreted. The hinge mechanism is easily rotated, pushing the silicone sheet in a smooth motion, opening the valve 20 with a larger hole diameter for excreting waste easily.

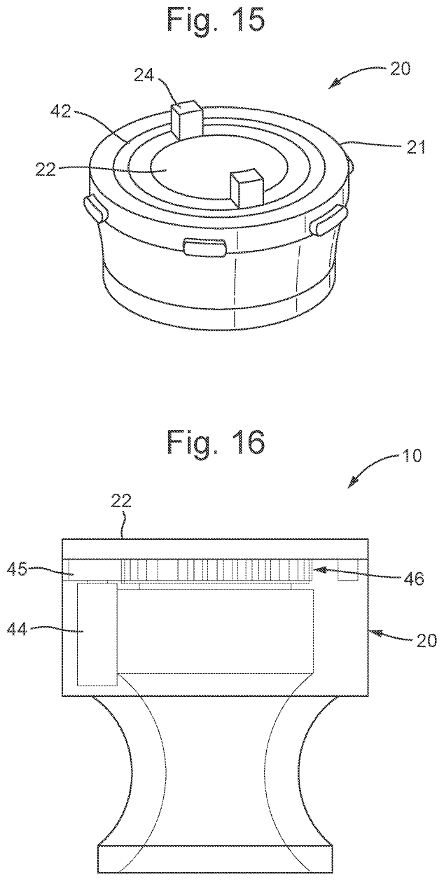

FIG. 15 is a perspective view of a valve 20 having an alternative valve mechanism. The valve mechanism may be called a flex-valve mechanism. As shown in FIG. 15, optionally the valve 20 comprises a tube 21, which may be flexible. The valve 20 further comprises a cap ring 22. The cap ring 22 may correspond to the external ring 22 described above. The cap ring 22 comprises a handle 24 for rotating the cap ring 22. Rotation of the cap ring 22 controls the valve 20 between the open position and the closed position.

Although not shown in FIG. 15, the valve 20 may comprise a cap. The cap is attached (e.g. glued) to the tube 21 and to the cap ring 22. The cap ring 22 is configured to rotate relative to the tube 21. When the cap ring 22 is rotated relative to the tube 21, the cap is controlled between a folded configuration corresponding to the open positon and a planar configuration corresponding to the closed position. In the planar configuration, the cap may substantially cover the whole of the opening defined by the cap ring 22 and the tube 21. As the cap ring 22 is rotated relative to the tube 21, the cap begins to fold. The cap is made of a flexible material allowing it to fold. When the cap folds, the passage through the valve 20 is opened up, thereby allowing waste material to pass through the valve 20 adjacent to the cap.

As shown in FIG. 15, optionally the valve 20 comprises a sealing ring 42. The sealing ring 42 may provide a seal between the cap ring 22 and the tube 21 so as to reduce waste material passing between the cap ring 22 and the tube 21.

The flex-valve mechanism is configured such that when a stool is pushing against the valve, the cap will provide firmer seal. Once the patient wants to excrete the stool, the cap can be folded by rotation of the mechanism, allowing easy waste excretion and cleaning.

The apparatus 10 may have a diameter of approximately 20 mm in the case of an ileostomy and 30 mm for a colostomy.

FIG. 16 is a schematic view of the valve 20. As shown in FIG. 16, optionally the valve 20 comprises an actuator for controlling the valve 20 between the open position and the closed position. The actuator may be controlled electronically and/or remotely.

As shown in FIG. 16, optionally the apparatus 10 comprises a motor 44. As shown in FIG. 16, the motor 44 may be part of the valve 20. This allows the motor 44 to be removed together with the rest of the valve 20, without surgery. Alternatively, the motor 44 may be part of the implant 11. As shown in FIG. 16, optionally the motor 44 is connected to a system of gears. The gears are for controlling the valve 20 between the open position and the closed position. For example, a first gear 46 may be rotated so as to actuate the mechanism (e.g. the iris mechanism, the hinge mechanism, the cap mechanism of FIG. 15 or the twisting mechanism of FIG. 10). The first gear 46 may be actuated by the second gear 45. The second gear 45 may be connected to the motor 44.

Electronic and/or remote control of the valve 20 may reduce the possibility of accidental damage to the valve 20 which may make it more convenient for the patient to operate the apparatus 10. The robotic actuation version of the valve 20 allows the patient to control when and how to open the valve 20 for releasing waste material or gas. Optionally all the electronics, including sensors, microcontrollers and batteries are embedded into a material such as silicone within the valve 20 during the manufacturing process. The battery can be either long lasting or rechargeable battery if connected with wireless recharging.

Optionally, the valve 20 comprises at least one sensor for measuring at least one physical property. For example, the valve 20 may comprise a temperature sensor for measuring temperature, for example the temperature of the waste material or the J-pouch. Additionally or alternatively, the valve 20 may comprise a pressure sensor for measuring pressure, for example, pressure of the waste material or the J-pouch. Additionally or alternatively, the valve 20 may comprises a pH sensor configured to measure the pH of the waste material or the J-pouch, for example. Additionally or alternatively, the valve 20 may comprise a water percentage (or humidity) sensor for measuring the water percentage (or humidity) of the waste material, for example. Additionally or alternatively, the valve 20 may comprise a hydration sensor for measuring the hydration of the bowel tissue, for example. Additionally or alternatively, the valve 20 may comprise a nerve electric signals sensor for measuring nerve electric signals. Optionally, the sensors are in data communication with one or more external devices. For example, the patient may check the sensors readings through a mobile application for easier stool maintenance and for notifying the patient of when they need to release a stool.

The smart apparatus 10 may be composed of hardware and software. The hardware is composed of a fixed portion that is implanted in the abdominal region (the implant 11) and the removable portion that is the actual valve (the valve 20). The software can also warn the patient if there is any other health condition arising or if hydration level has increased or lowered excessively.

As shown in FIG. 17, the apparatus 10 may be attached to the body part 30. The attachment may be made in a surgical operation. The implant 11 may be surgically attached to the body part 30. Such a surgical operation may further comprise a step of forming J-pouch in an intestine. The J-pouch may be for accumulating waste material to be released from the body. Once the implant 11 is attached to the body part 30, the valve 20 may be inserted into the implant 11 such that the implant 11 locks the valve 20 inside it. The apparatus 10 is suitable for connecting to any of the colon, the small intestine and the urethra. The apparatus 10 is suitable for use as an anal prosthetic.

The implant 11 may be recessed compared to the skin layer so that a soft stoma patch can cover it. The soft patch may be configured to interlock with the interior of the implant 11 or the valve 20 to allow for sealing that valve away from the sight, creating an almost seamless patch. The patch can be removed via a mechanical connection such as a flexible hinge or a clip. The patch can be made of a self-sticking material such as self-sticking silicone which may directly stick to the valve 20 or the implant 11. The patch can be decorated with tattoos and/or may comprise one or more LEDs, switches, pressure buttons and an electronic display.

By using the apparatus 10, the bowel 31 may not be required to be stitched onto the skin 32 but rather may be fixed onto the implant 11 inside the abdominal wall. Also, the stool collection is not external to the body. An advantage of the apparatus 10 is reducing physical and psychological burden of stoma patients by reducing leakage, reducing production of bad odour, and/or reducing the probability of skin blistering and infection.

The valve 20 may be completely removable for deep cleaning and disinfection and easy periodic maintenance. Known devices cannot be easily removed for maintenance and it is not possible for them to last in some cases more than 50 years (in the case of young patients). The apparatus 10 described herein has mechanical and electronic components with a limited lifespan moulded in the same unit that can be easily inserted and removed by trained clinical staff or the patients themselves. Being modular in nature the apparatus 10 can be provided in different modes with different level of technologies.

Optionally, the apparatus 10 has a low-profile in use. The implant 11 may be flush with the body. The apparatus 10 may be implanted in lieu of the anus for those patients who lost it to bowel cancer or faecal incontinence, for example.

The apparatus 11 can be used by colorectal surgeons for an ostomy surgery such as colostomy, ileostomy and urostomy to eliminate the need of a stoma bag for waste accumulation. Stoma surgeries may be replaced, or previously generated stomas may be reconverted by using the apparatus 10.

1. A prosthetic sphincter apparatus comprising:
a valve controllable between an open position for allowing waste to pass through the valve and a closed position for preventing waste from passing through the valve; and
an implant configured to be surgically attached to a body part and to lock the valve inside the implant,
wherein the valve is configured to be controllable between the open position and the closed position while remaining locked inside the implant.

2. The prosthetic sphincter of claim 1, wherein the valve and the implant are configured such that the valve can be repeatedly removed from and reinserted into the implant.

3. The prosthetic sphincter of claim 1, wherein a plurality of holes are defined in the implant for allowing the implant to be fixed to the body part.

4. The prosthetic sphincter of claim 1, wherein the implant comprises a flange protruding radially outwards for anchoring the implant.

5. The prosthetic sphincter of claim 4, wherein the flange comprises a mesh.

6. The prosthetic sphincter of claim 1, wherein the implant comprises external ridges for gripping the implant to tissue.

7. The prosthetic sphincter of claim 1, wherein the implant comprises hooks for allowing the implant to be fixed to muscle.

8. The prosthetic sphincter of claim 1, wherein the implant comprises a skirt for anchoring the implant to the peritoneum.

9. The prosthetic sphincter of claim 1, wherein the valve comprises a flexible tube defining a channel and arranged such that twisting of one end of the flexible tube relative to the other end of the flexible tube causes the valve to be controlled between the open position in which the channel is open and the closed position in which the channel is at least partly closed.

10. The prosthetic sphincter of claim 9, wherein the valve comprises an external ring attached to one end of the flexible tube and configured such that rotation of the external ring causes the end of the flexible tube to which the external ring is attached to be rotated relative to the other end.

11. The prosthetic sphincter of claim 9, wherein the valve comprises an internal ring attached to one end of the flexible tube and configured to be fixed relative to the implant when the valve is locked inside the implant.

12. The prosthetic sphincter of claim 9, arranged such that the ends of the flexible tube can be rotated at least one full rotation relative to each other.

13. The prosthetic sphincter of claim 12, arranged such that the ends of the flexible tube can be twisted at least two full rotations relative to each other.

14. The prosthetic sphincter of claim 1, wherein the valve comprises moveable components configured to be moveable between a first position corresponding to the open position and a second position corresponding to the closed position in which the moveable components at least partly block a channel through the valve.

15. The prosthetic sphincter of claim 14, wherein the moveable components comprise blades of an iris mechanism.

16. The prosthetic sphincter of claim 14, wherein the moveable components comprise hinged rods of a hinge mechanism.

17. The prosthetic sphincter of claim 1, wherein the valve comprises a tube, a cap ring and a cap attached to the tube and the cap ring, wherein the cap ring is rotatable relative to the tube so as to control the cap between a folded configuration corresponding to the open position and a planar configuration corresponding to the closed position.

18. The prosthetic sphincter of claim 1, wherein the valve comprises a handle for manually controlling the valve between the open position and the closed position.

19. The prosthetic sphincter of claim 1, wherein the valve comprises an actuator for controlling the valve between the open position and the closed position.

20. The prosthetic sphincter of claim 1, wherein the valve comprises at least one sensor for measuring at least one physical property.

21. The prosthetic sphincter of claim 20, wherein the at least one physical property comprises temperature, pressure, pH, hydration, humidity and/or nerve electric signals.

22. The prosthetic sphincter of claim 1, wherein the valve comprises parts that are moveable relative to each other.

23. The prosthetic sphincter of claim 1, wherein the outer diameter of the implant is greater than the outer diameter of the valve.

24. The prosthetic sphincter of claim 1, wherein the implant surrounds the valve when the valve is locked inside the implant.

25. The prosthetic sphincter of claim 1, wherein the implant comes in two separate pieces, one of the two separate pieces being an attachment assembly for attaching to the body part and the other of the two separate pieces being a main body into which the valve can be locked.

26. A method for attaching a prosthetic sphincter apparatus, the method comprising:
fixing an implant to a body part, the implant configured to lock a valve inside the implant, the valve being controllable between an open position for allowing a stool to pass through the valve and a closed position for preventing waste material from passing through the valve, wherein the valve is configured to be controllable between the open position and the closed position while remaining locked inside the implant.

27. The method of claim 26, wherein the fixing comprises:
surgically attaching an attachment assembly of the implant to the body part.

28. The method of claim 26, wherein the fixing comprises:
non-surgically attaching a main body of the implant to an attachment assembly that has already been attached to the body part.

29. The method of claim 26, comprising:
forming a J-pouch in an intestine for accumulating waste material to be released from the body.

30. The method of claim 26, comprising:
inserting the valve into the implant such that the implant locks the valve inside it.
